(19) United States
(12) Patent Application Publication
(10) Pub. No.: US 2006/0223195 A1
(43) Pub. Date: Oct. 5, 2006

(54) Stress based removal of nonspecific binding from surfaces

(76) Inventors: Grant D. Meyer, Ithaca, NY (US); Jose Manuel Moran-Mirabal, Ithaca, NY (US); Harold G. Craighead, Ithaca, NY (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/280,942

(22) Filed: Nov. 16, 2005

Related U.S. Application Data
(60) Provisional application No. 60/628,322, filed on Nov. 16, 2004.

(51) Int. Cl. G01N 33/543

(52) U.S. Cl. 436/518

(57) ABSTRACT

A biological material sensing surface is exposed to a biological material that is selectively bound to a selected sensing portion of the sensing surface. The sensing surface is then subjected to shear stress oscillations to selectively remove nonspecifically bound material. The shear stress may be provided by an ultrasound resonator operating at a power sufficient to selectively remove nonspecifically bound biological material, such as protein from non-sensing areas of the sensing surface, which may be micropatterned array.

### RELATED APPLICATION

This application claims the benefit of United States Provisional Application Ser. No. 60/628,322 (entitled Nonspecific Binding Removal from Protein Microarrays Using Thickness Shear Mode Resonators, filed Nov. 16, 2004) which is incorporated herein by reference.

### GOVERNMENT FUNDING

The invention described herein was made with U.S. Government support under Grant Number MDA-972-00-1-0021 awarded by DARPA. The United States Government has certain rights in the invention.

Use may have been made of the Sandia laboratory. Sandia is a multi-program laboratory operated by Sandia Corporation, a Lockheed Martin Company, for the United States Department of Energy's National Nuclear Security Administration under contract DEAC0494AL85000.

### BACKGROUND

Nonspecific binding decreases bioassay sensitivity, specificity, and reproducibility, which limit optical, electrochemical, and gravimetric biosensors, and can alter statistical analyses performed on microarrays. While appropriate surface chemistry may reduce nonspecific binding on non-sensing areas, this chemistry cannot be applied to sensing areas where specific binding occurs. These areas can nonspecifically bind solution components leading to an inflated, falsely positive signal. Alternatively, nonspecific binding to non-sensing control areas reduces sensitivity, leading to false negatives. It is noted that the literature does not clearly distinguish between the terms “nonspecific”, “promiscuous”, “fouling”, and “cross-reactivity”. The term nonspecific is meant to encompass each of those terms along with other synonyms.

Antibody aggregates also create experimental difficulties in microarray processing. Producing aggregation resistant antibodies may reduce aggregate formation, but requires additional time and cost. Nondestructive nonspecific binding removal improves data quality, simplifies analysis, and increases assay fidelity.

### SUMMARY

A surface is exposed to a biological material that is selectively bound to selected sensing portions of the surface. The surface is then subjected to mechanical or shear stress to selectively remove nonspecifically bound material.

In one embodiment, the stress is provided by an ultrasound resonator operating at a power sufficient to selectively remove nonspecifically bound protein from non-sensing areas of the surface, such as a micropatterned protein array.

In a further embodiment, the surface comprises a quartz crystal resonator having a gold layer with patterned protein squares. A mechanical stress is generated that appears to reduce the activation energy of desorption, expediting nonspecifically bound protein removal. Various power levels and frequencies may be used to remove nonspecifically bound protein and other contaminants (e.g. dust)

### BRIEF DESCRIPTION OF THE DRAWINGS

FIG. 1 is a block schematic diagram of a resonator having biosensing array formed thereon according to an example embodiment.

FIG. 2 is a schematic diagram illustrating initial surface chemistry for the resonator of FIG. 1 according to an example embodiment.

FIG. 3 is a schematic diagram illustrating surface chemistry schematic after resonator activation according to an example embodiment.

FIG. 4A illustrates fluorescent intensity from sensing squares vs. time at three power levels according to an example embodiment.

FIG. 4B illustrates fluorescent intensity from non-sensing area vs. time. according to an example embodiment.

FIG. 4C illustrates average fluorescent square intensity divided by non-sensing area average intensity vs. time plot at 3.5 W and 14 W resonator power levels according to an example embodiment.

FIG. 5A illustrates 3D fluorescent intensity plot demonstrating aggregate intensity compared to pattern intensity before QCR operation according to an example embodment.

FIG. 5B illustrates 3D fluorescent intensity plot demonstrating uniform pattern fluorescent intensity after QCR operation (3.5 W, 20 min, pH 4) according to an example embodiment.

FIG. 6 is a photograph of a QCR flow cell with integrated fluidics and electrical connections.

FIG. 7A illustrates fluorescent intensity image after pixel intensity discrimination and conversion to logical array according to an example embodiment.

FIG. 7B illustrates fluorescent intensity image after pixel intensity discrimination, areal discrimination, and conversion to logical array according to an example embodiment.

FIG. 8 illustrates various surface chemistries during a process of capturing and removing biological materials.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description is, therefore, not to be taken in a limited sense, and the scope of the present invention is defined by the appended claims.

Routinely, resonators have been used as ultra-sensitive mass detectors, and are typically referred to as quartz crystal microbalances. In one embodiment of the present invention, compact, reliable quartz crystal resonators may be used to remove nonspecific binding from surfaces, and improve fluorescent biosensor signal accuracy. Piezoelectric transducers may also be used to vibrate surfaces at various frequencies to remove nonspecific binding. Any transducer capable of generating shear stress near a selected surface having nonspecific binding may be used.

FIG. 1 is a block schematic diagram of a resonator 100 having biosensing array formed thereon according to an example embodiment. While a biosensing array is described, any surface binding assay may be used. Binding assays including aptimers for example. Antibodies, DNA or other binding recognition molecules may be used on various surfaces and may be formed in individual shapes or arrays.

In one embodiment the resonator 100 is formed of quartz, and a layer of gold is deposited on it to provide a binding site for various proteins. The biosensing surface is represented by an array of squares 110 corresponding to patterned protein sensing areas. Non sensing areas are the space 115 between the sensing areas 110. Electrical contacts 120, 125 may be used to actuate a piezzoelectrically driven QCR in one embodiment. Other forms of actuation may also be used.

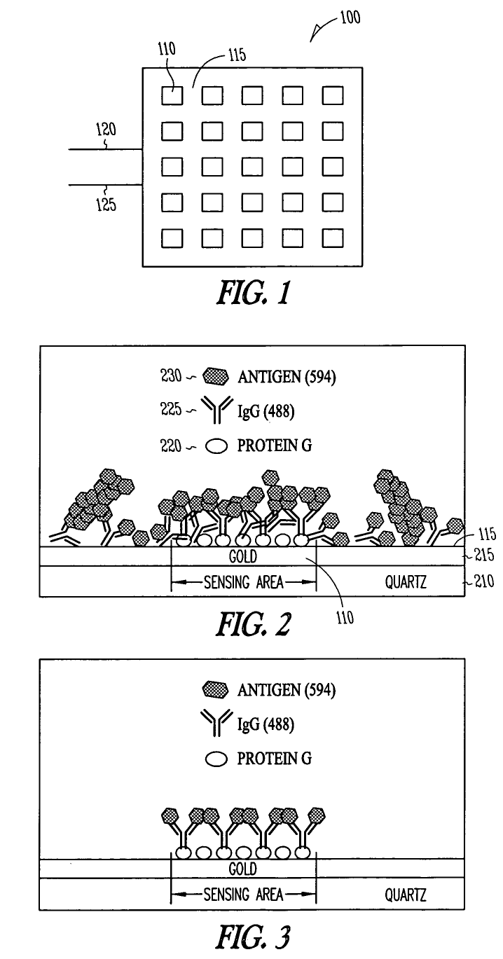

FIG. 2 is a schematic diagram illustrating initial surface chemistry for the resonator of FIG. 1. The quartz resonator is indicated at 210, and the gold layer is indicated at 215. Symbols used to represent protein G at 220, IgG (488) at 225 and antigen (594) at 230 are shown in a table in the figure. To create model micropatterned surfaces having both specifically and nonspecifically bound protein, QCRs may be coated with parylene-C, photolithographically patterned, and etched in a common manner. Protein G 220 may then be covalently linked to lithographically defined gold areas 215, and parylene-C removed, leaving patterned protein G squares 110, and corresponding to the protein G symbols in FIG. 2.

The patterned protein G squares 110 measuring 20×20 μm in one embodiment, define sensing areas 110. A single sensing area may also be used, as well as many different types of sensing molecules or other mechanisms to create surface binding. The surrounding area defines the non-sensing control area 115 in FIG. 1. Fluorescently tagged antibody (IgG goat anti-mouse) 225 and antigen (IgG mouse anti-rabbit) 230 may be added in succession to demonstrate operation of the biosensing array. Shear stress, provided by oscillating the resonator may be used to selectively remove nonspecifically bound protein G and immunoglobulins, while maintaining specifically bound antibody activity.

Shear wave penetration is thought to generate mechanical stress on bound materials to reduce the activation energy of desorption, which expedites nonspecifically bound material removal as illustrated in FIG. 3, wherein the numbering is consistent with FIG. 2. One possible way to calculate the wave penetration decay length, uses the following equation

$$δ = {( \frac{{η}_{L}}{π {f}_{0} {ρ}_{L}} )}^{1 / 2}$$

where η_{L} is the fluid viscosity, ρ_{L} is the fluid density, and f₀ is the fundamental frequency. For a 5 MHz resonator operated in buffer, δ=250 nm. In one model covalent linking system, the Stokes' radius for protein G is 3 nm, 5.5 nm for an IgG, and the covalent thiol linker is 1 nm long. The film thickness for a system with covalently bound protein G, antibody, and antigen should be about 29 nm, well within one decay length. Hence, the entire protein system becomes entrained, and a similar shear stress is present throughout the multilayer system. The equation may be used to develop parameters for may different arrays. Empirical methods may also be used to vary the parameters, such as frequency to select optimal parameters for different devices.

Micropatterns clearly defined sensing and non-sensing areas. The non-sensing area acted as a control for both fluorescence and AFM experiments. In one embodiment, digital image segregation of sensing and non-sensing areas may be achieved with a clearly defined pattern. Signal may be defined as fluorescent intensity from the sensing squares. Background may be defined as fluorescent intensity from the non-sensing area.

In one embodiment, a pH value of 4 may maintain specific antibody/protein G interactions and remove the most nonspecific binding during resonator operation. The F_{c} region of IgG has the highest affinity for protein G at pH 4. In one embodiment, fluorescent intensity values may be normalized after 3 mL of pH 4 PBS buffer is washed through the flow cell at 1 mL/min to remove fluid flow effects from data. FIG. 2 represents surface chemistry prior to piezo activation, and FIG. 3 represent surface chemistry after 20 min of resonation at 3.5 W input power.

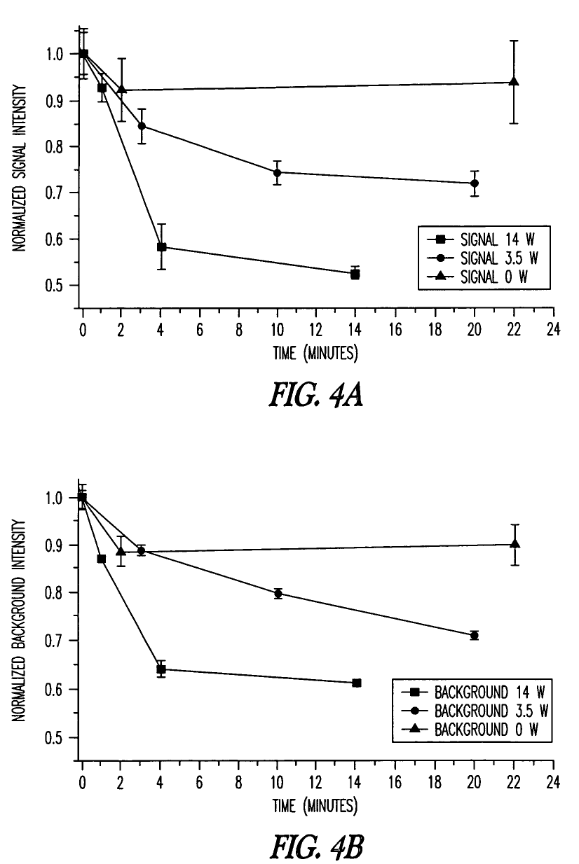

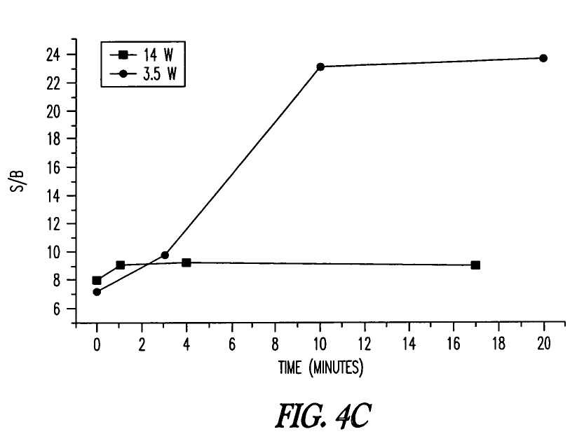

Images analyzed through various experiments demonstrated significant removal of non-specifically bound protein adsorbed to both the micropattemed protein sensing array and non-sensing surface. Average signal and background values from such experiments are plotted in FIGS. 4A-C. FIG. 4A illustrates fluorescent intensity from sensing squares vs. time at three power levels. Fluorescent intensity is from both 488 and 594 probes. Lines have been added to guide the eye, and fluorescent intensity standard deviation bars demonstrate fluorescence intensity non-uniformity in captured images. FIG. 4B illustrates fluorescent intensity from non-sensing area vs. time. FIG. 4C illustrates average fluorescent square intensity divided by non-sensing area average intensity vs. time plot at 3.5 W and 14 W resonator power levels according to an example embodiment.

In FIGS. 4A-C, intermediate data points were extracted from images not shown. Removal significantly improved sensing and non-sensing area fluorescent intensity uniformity. This result is evident in FIGS. 4A and 4B. With resonator operation, fluorescent intensity standard deviation values became progressively smaller compared to the control at 0 watts.

At low power levels (i.e. 3.5 W) nonspecific binding was removed primarily from non-sensing areas. Hence, the signal-to-background ratio value increased markedly. In contrast, such significant nonspecifically bound protein removal from sensing areas occurred at 14 W that signal-to-background values increased only marginally as illustrated in FIG. 4C. At 14 W the signal-to-background ratio remained constant after high power operation. This indicates that QCR operation sets an affinity threshold. Above this threshold, specifically bound antibodies with affinities greater than the removal stress exerted by the QCR were retained, while nonspecifically bound antibodies were removed. In further embodiments, power levels of at least approximately 3 W may be used. In still further embodiments, power levels may be significantly varied.

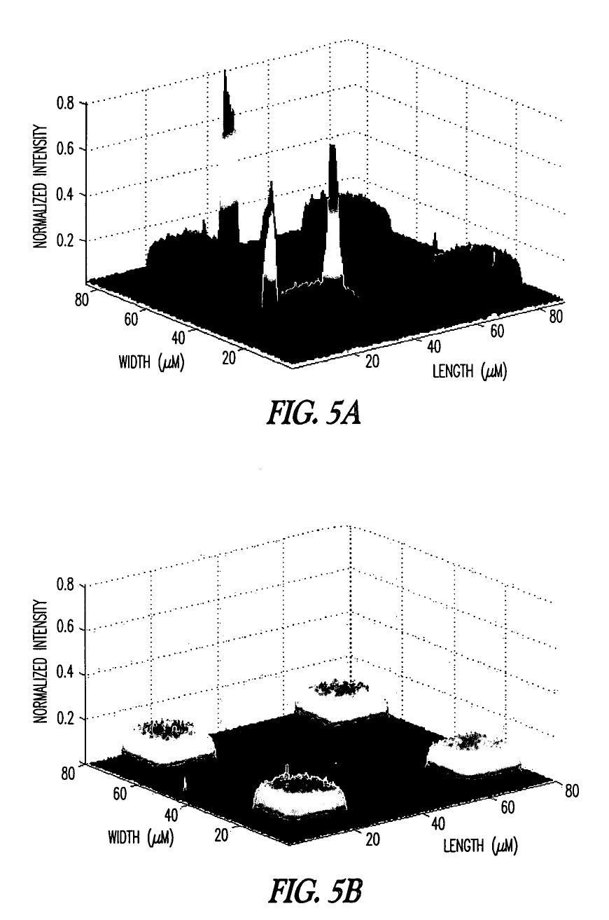

A constant signal-to-background ratio also indicates that the F_{c}-protein G and antibody-antigen binding interactions were maintained. Hence, after QCR operation, fluorescent intensity values resulting from specifically bound protein left after resonator operation accurately define the true signal. Pattern uniformity markedly improved, as demonstrated in FIGS. 5A and 5B, further validating the presence of only specifically bound species. FIG. 5A illustrates 3D fluorescent intensity plot demonstrating aggregate intensity compared to pattern intensity before QCR operation. FIG. 5B illustrates 3D fluorescent intensity plot demonstrating uniform pattern fluorescent intensity after QCR operation (3.5 W, 20 min, pH 4) according to an example embodiment.

Fluorescent intensity from nonspecifically bound protein on non-sensing areas dropped by more than 85% and by 77% on sensing squares after resonator operation at 14 W, corresponding well with the AFM film thickness reduction observations. Fluorescent intensity drops reported include nonspecific binding removal with fluid flow.

The example data provided herein may vary with different arrays, and is only represented as one example of data that may be obtained. Other data will likely be obtained with other experiments and examples.

### EXAMPLES

One example QCR operating at 5 MHz is generally available and was obtained from Maxtek, Inc. Resonators were washed with acetone, isopropanol, and dried under nitrogen. Poly-ethylene oxide at a concentration of 0.1% in distilled water was spun on the resonators. Parylene-C was deposited to a thickness of 1.5 μm +/−0.1 μm (SCS-Cookson). Positive tone Shipley photoresist (1827) was spun over the parylene-C film at 2000 rpm, and soft baked at 90° C. for 60 seconds. A contact mask with 20 μm squares was used to define features in the photoresist. AZ 300 MIF developer defined squares, which were then etched in an oxygen plasma. Care was taken to ensure all parylene in etched regions was removed, but little gold was sputtered. After micropatterning, photoresist was removed using acetone, isopropanol, and dried under nitrogen.

Dithiobis[succinimidylpropionate] (DSP) was used to covalently link amines of protein G to open gold areas (Pierce Biotechnology, Inc.). Instructions were followed according to manufacturer specification with a five minute sonication step and 20 second centrifugation at 2000 rpm being the only additions to the protocol. These steps were added to ensure saturation and excess DSP pellet formation respectively. Protein G was used to properly orient the F_{c} region of IgG towards the gold surface leaving the F_{ab} regions to bind antigen. Protein G was incubated at a concentration of 1 mg/mL for two to four hours prior to washing.

After covalent protein G linkage to the resonator surface, the parylene-C layer was peeled from the resonator leaving the patterned protein G surrounded by the original gold electrode. Antibodies were labeled with Alexa Fluor 488 and Alexa Fluor 594 respectively following the Molecular Probes protocol. Antibodies (polyclonal IgG goat anti-mouse (H+L)) and antigen (polyclonal IgG mouse anti-rabbit (H+L)) were then added in successive two to four hour incubation steps at 200 μg/mL.

Each resonator was washed three times after each incubation step. Fluorescent intensity images were obtained after rigorous washing. FIG. 6 is a photograph of a QCR flow cell 600 with integrated fluidics and electrical connections. The flow cell was machined out of two polycarbonate pieces (lid and base). A silicone seal was cast into the machined lid, and silicone tubing was cured into the silicone seal of the lid. The bottom half was machined to accept pogo pins for electrical contact.

Resonators were kept wet at all times prior to insertion into the flow cell. The flow cell was formed for convenient electrical and fluidic connection to each resonator, as well as in situ observation, while still allowing repeated removal for quantitative imaging. Flow cell volume was 250 μL. Many different types of enclosures and fluid delivery systems may be used. In one embodiment, the enclosure is formed to allow easy insertion and removal of resonators for analysis. Further embodiments may utilize integrated optical detection systems for determining the presence of bound biological materials.

A resonator input was generated by an Agilent (SA4402B) spectrum analyzer and amplified with an ENI 325LA broadband power amplifier. After liquid loading, each resonator was scanned over a large span to find the resonant frequency near 5 MHz. In further embodiments, other frequencies may also be used. Center frequency adjustment and span reduction provided a relatively constant drive amplitude near resonance. Note that in one embodiment, the span was not set to zero because mass desorption and temperature fluctuations shift the resonant peak. To account for these shifts, the analyzer was set to auto-track the resonant peak. Power delivered to a QCR was determined by measuring the return loss of the resonators and subtracting from the amplified output power.

Prepared resonators were imaged with a 20× NA 0.7 water immersion objective prior to placement in the flow cell. Images were taken near the center (active area) of each resonator, and all images were taken after removal from the flow cell. Photobleaching was observed during prolonged exposure; for accurate quantitation, the number of exposures was minimized. Quantitated images were taken in RGB mode with gain 8 and exposure times of 400 msec (488 nm) and 200 msec (594 nm) with an Olympus A×70 microscope and SPOT RT CCD. A filter cube transmitting fluorescence at both wavelengths (488 nm and 594 nm) was used to capture images without excessive photobleaching. Critical to accurate background quantitation, gamma was always defined to be one, so as not to bias the image towards high intensity or low intensity pixels. Images were taken at 1520×1080 pixel resolution, rotated, and cropped to approximately 600×900 pixels. Image cropping was used to reduce systematic nonuniform illumination error. Rotation may be performed prior to analysis to ensure algorithm fidelity.

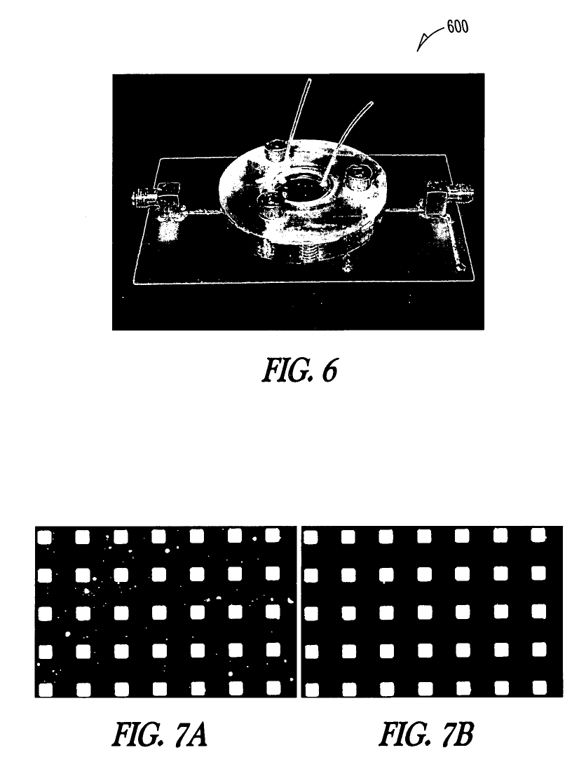

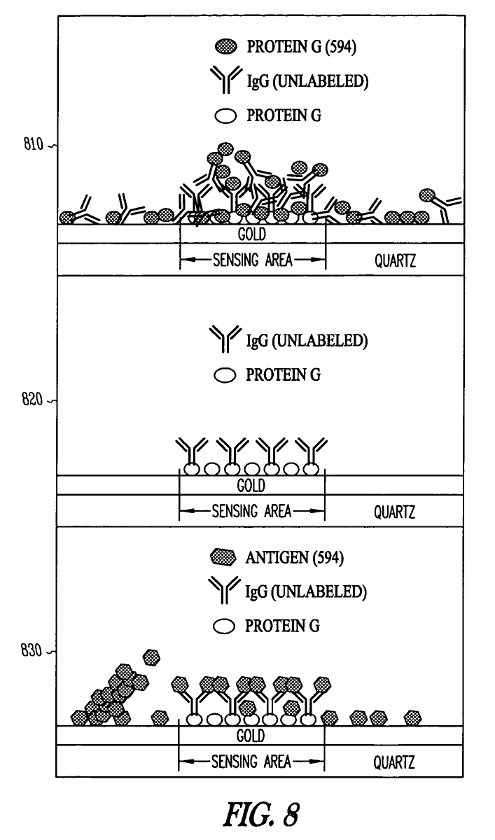

Image analysis code was written to discriminate between signal and background pixels. Complicating matters in intensity thresholding was nonspecific protein binding and protein aggregation. Aggregates, ranging from nanometers to microns, bind strongly to both nonpatterned and patterned areas. Since a thresholding method based solely on intensity associates these bright particles as signal, the signal is improperly inflated and background deflated. FIG. 7A illustrates fluorescent intensity image after pixel intensity discrimination and conversion to logical array according to an example embodiment. FIG. 7B illustrates fluorescent intensity image after pixel intensity discrimination, areal discrimination, and conversion to logical array according to an example embodiment.

Arrays were used to compute the average signal, background, signal-to-background and standard deviation values. Statistics were generated from 540,000 pixel populations. AFM measurements were made in tapping mode. Devices were dried under nitrogen, and scanned with silicon nitride cantilevers (Veeco).

To further confirm nonspecifically bound protein removal from patterned sensing areas, a resonator was patterned with nonfluorescent covalently bound protein G, followed by washing, parylene-C film removal, and incubation with nonfluorescent IgG goat antimouse. The resonator was then incubated for four hours with Alexa 594 labeled protein G and washed. If the F_{c} region of each bound antibody is attached to the covalently bound (unlabeled) protein G, fluorescently tagged protein G should not bind to patterned areas to a greater degree than the non-sensing control area. FIG. 8 illustrates various surface chemistries during a process of capturing and removing biological materials. Prior to resonation, as represented by surface chemistry 810, the observed pattern is highly visible and brighter than the background. Protein G appears to have bound nonspecifically to IgG goat antimouse and possibly to excess (unlabeled) protein G.

After resonator operation 820 at 24.7 W, nonspecifically bound fluorescent and non-fluorescent protein (i.e. 594 labeled protein G and unlabeled IgG goat anti-mouse) were removed. The maximum input power of 24.7 W was used in later experiments to verify that antibody film integrity was maintained at maximum power and to ensure that fluorescent intensity values after QCR operation at 14 W matched higher power operation fluorescent intensity values. Comparable fluorescent intensity signal values were obtained after QCR operation at both 14 W and 24.7 W, which indicated that equivalent nonspecific binding protein quantities were removed at both 14 W and 24.7 W.

To eliminate the possibility that specifically bound IgG goat anti-mouse was removed and the antigen bound directly to the covalently bound protein G, a resonator was prepared with IgG goat anti-mouse labeled with Alexa 488. After operation (24.7 W, 2 min, pH=4). It was evident that the pattern was uniform and the IgG goat anti-mouse capture layer was still present.

Adding Alexa 594 labeled antigen (IgG mouse anti-rabbit) demonstrated that the specifically bound IgG goat anti-mouse (unlabeled), bound to the patterned protein G squares, was still active after high shear as illustrated at 830.

Resonator operation removed nonspecifically bound protein and aggregates on all areas. To ensure that only nonspecifically bound protein removal occurred, atomic force microscope (AFM) images were obtained using dried resonators. No resonator was operated after drying. Three separate resonators were imaged, two before, and one after operation. The first image was taken with a resonator prepared with patterned protein G, IgG goat anti-mouse, and antigen. Parylene was removed prior to IgG and antigen incubation steps. The pattern is visible, but appears blanketed by nonspecifically bound protein layers, and large protein aggregates.

To determine the absolute pattern height, the entire protocol (linker, protein G, IgG goat anti-mouse, and antigen) was repeated without removing parylene until the end. Washing steps were performed after each incubation step and after parylene removal. The film thickness was much greater than the expected 29 nm, indicating that multiple layers existed on the patterned sensing areas.

Another resonator was prepared as described above and operated at high power (24.7 W, 2 minutes, pH=4). This power level significantly reduced pattern intensity. Contrary to what might be expected, the film was not sheared from the surface, but in fact, a film thickness much closer to 29 nm was found. Intensity data combined with AFM results indicated that film uniformity was significantly improved after QCR operation. At this power, sensing area chemistry accurately matched the intended chemistry, not a mixture of specifically and nonspecifically bound antibody.

At pH 2.8 protein G/IgG interactions are disrupted. To explore additional purification and preconcentration applications, buffer was switched from the incubation buffer (pH 7.4) to pH 2.8 with the resonator operating at 1.8 W. Rapid antibody elution resulted. After five minutes the resonator was removed and imaged. Both nonspecifically and specifically bound protein were removed with 94% efficiency. Hence, QCRs could be used to purify antigen and later release it for downstream analysis. The binding surface may also be reused if desired. Higher operating powers may also facilitate removal of both nonspecifically and specifically bound protein for potential binding surface reuse.

### CONCLUSION

Biosensors and bioassays should ideally be fast, simple, and accurate. Most importantly, neither false positives nor false negatives should result. Nonspecific binding can create false signal, or mask true signal. It also increases assay variability and decreases assay accuracy. Nonspecific binding may be removed and assay reproducibility and signal validity improved. Results confirm quartz crystal resonator operation increases pattern uniformity and simplifies data analysis. This problem is chemically intractable on areas with sensing molecules, and hence, this mechanical approach should prove valuable for high sensitivity/specificity bioassays, protein-protein interaction studies, library screening, purification, and biosensors. The power levels used in the examples may vary significantly in various embodiments. Other transducers may achieve similar results at significantly lower power levels and at different frequencies. Lower power levels may be desirable when considering portability.

The Abstract is provided to comply with 37 C.F.R. §1.72(b) to allow the reader to quickly ascertain the nature and gist of the technical disclosure. The Abstract is submitted with the understanding that it will not be used to interpret or limit the scope or meaning of the claims.

1. A method comprising:
exposing a biological material sensing surface to biological material; and
subjecting the surface to shear stress to selectively remove nonspecifically bound biological material.

2. The method of claim 1 wherein the shear stress is provided by a resonator.

3. The method of claim 2 wherein the resonator is operated at a power of at least approximately 3 W for a selected period of time.

4. The method of claim 2 wherein the resonator is operated at a power of at least approximately 14 W.

5. The method of claim 2 wherein the resonator comprises a quartz crystal resonator.

6. The method of claim 1 wherein the shear stress is provided by ultrasonic waves.

7. The method of claim 1 wherein the shear stress is provided by an ultrasound resonator operating at a power sufficient to selectively remove nonspecifically bound protein from non-sensing areas of the sensing surface.

8. The method of claim 1 wherein the sensing surface comprises a micropatterned array.

9. The method of claim 1 wherein the shear stress is provided by an ultrasound resonator operating at a power sufficient to selectively remove nonspecifically bound protein from sensing surface.

10. The method of claim 1 wherein the sensing surface comprises protein sensing areas and non-sensing areas.

11. A method comprising:
exposing a biological material sensing surface to biological material, which binds at sensing portions of the sensing surface; and
subjecting the sensing surface to ultrasonic waves to selectively remove nonspecifically bound biological material.

12. A method comprising:
exposing a protein array to proteins;
subjecting the protein array to shear stress to selectively remove nonspecifically bound protein; and
detecting florescent intensity from the array.

13. A method comprising:
creating a sensing area on a quartz crystal resonator;
exposing the sensing area to labeled biological material;
subjecting the sensing area to shear stress by oscillating the quartz crystal resonator to selectively remove nonspecifically bound biological material; and
detecting florescent intensity from the sensing area.

14. The method of claim 13 and further comprising washing the sensing area prior to detecting florescent intensity.

15. The method of claim 13 wherein the shear stress is provided by driving the resonator at approximately its resonant frequency.

16. The method of claim 15 wherein the resonant frequency is approximately 5 MHz.

17. The method of claim 13 wherein the protein sensing area is formed steps comprising:
coating the quartz crystal oscillator with a layer of gold;
masking the layer of gold;
forming patterned sensing squares on unmasked areas of the layer of gold; and
removing the mask.

18. The method of claim 17 wherein the biological material comprises protein G.

19. The method of claim 17 wherein the mask comprises parylene-C.

20. The method of claim 17 wherein the sensing squares are approximately at least 20×20 μm in size.
